(19) United States
(12) Patent Application Publication
(10) Pub. No.: US 2024/0001198 A1
(43) Pub. Date: Jan. 4, 2024

(54) METHOD, DEVICE, AND COMPUTER READABLE STORAGE MEDIUM FOR IMPROVING PHYSICAL PERFORMANCE

(71) Applicant: bOMDIC, Inc., New Taipei (TW)

(72) Inventors: PIN-YU CHEN, New Taipei (TW); CHUNG-YEN WU, New Taipei (TW)

(21) Appl. No.: 17/853,963

(22) Filed: Jun. 30, 2022

(51) Int. Cl. A63B 24/00

(52) U.S. Cl. CPC A63B 24/0075; A63B 2024/0068; A63B 2230/75

(57) ABSTRACT

A method, a device, and a computer readable storage medium for improving the physical performance of the user are provided. The method includes: determining a reference value of the physical performance of the user in a first reference time point of a physical activity duration and a final target value of the physical performance corresponding to a final target time; determining a plurality of middle target values of the physical performance arranged in increasing order in the physical activity duration based on the reference value, the final target value, and the final target time, wherein the plurality of middle target values of the physical performance respectively corresponds to a plurality middle target times; and providing a physical activity suggestion to gradually meet the target values of the physical performance.

### BACKGROUND OF THE INVENTION

### 1. Field of the Invention

The disclosure relates to a method, device, and computer readable storage medium for improving physical performance of a user, and more particularly for improving the physical performance of the user to gradually meet a plurality of target values of the physical performance.

### 2. Description of Related Art

Most people appreciate the importance of physical fitness. Usually, users would set up a target of the physical performance and want to improve the physical performance to meet the target. However, most users usually do not know how to set an appropriate target, such as a target that is achievable within a specific time period.

Therefore, if it is able to design a technical solution that may help the user to set up or adjust the target of the physical performance, it helps the user to improve the physical performance according to the target step by step.

### SUMMARY OF THE INVENTION

The disclosure is directed to a method, device, and computer readable storage medium for improving the physical performance of a user, which are adapted to resolve the aforementioned technical problems.

An embodiment of the disclosure provides a method for improving the physical performance of a user, which is adapted to a device, and includes: determining a reference value of the physical performance of the user in a first reference time point of a physical activity duration and a final target value of the physical performance corresponding to a final target time; determining a plurality of middle target values of the physical performance arranged in increasing order in the physical activity duration based on the reference value, the final target value, and the final target time, wherein the plurality of middle target values of the physical performance respectively corresponding to a plurality middle target times; and providing a physical activity suggestion to gradually meet the target values of the physical performance.

An embodiment of the disclosure provides a device for improving the physical performance of a user, which includes a storage circuit and a processor. The processor is coupled to the storage circuit, and accesses the program code to: determine a reference value of the physical performance of the user in a first reference time point of a physical activity duration and a final target value of the physical performance corresponding to a final target time; determine a plurality of middle target values of the physical performance arranged in increasing order in the physical activity duration based on the reference value, the final target value, and the final target time, wherein the plurality of middle target values of the physical performance respectively corresponding to a plurality middle target times; and provide a physical activity suggestion to gradually meet the target values of the physical performance.

An embodiment of the disclosure provides a computer readable storage medium, the computer readable storage medium records an executable computer program, and the executable computer program is loaded to a s device for improving the physical performance of a user to perform following steps: determining a reference value of the physical performance of the user in a first reference time point of a physical activity duration and a final target value of the physical performance corresponding to a final target time; determining a plurality of middle target values of the physical performance arranged in increasing order in the physical activity duration based on the reference value, the final target value, and the final target time, wherein the plurality of middle target values of the physical performance respectively corresponding to a plurality middle target times; and providing a physical activity suggestion to gradually meet the target values of the physical performance.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 illustrates a schematic block diagram of an exemplary device in the present invention.

FIG. 2 illustrates an embodiment of the physical activity duration when the method for improving the physical performance of the user is applied.

FIG. 3 illustrates an embodiment of the modified physical activity duration 31 when the modified method for improving the physical performance of the user is applied.

### DETAILED DESCRIPTION OF THE ILLUSTRATED EMBODIMENTS

The detailed explanation of the present invention is described as following. The described embodiments are presented for purposes of illustrations and description, and they are not intended to limit the scope of the disclosure.

In the present disclosure, the term “physical performance” refers to the energy expenditure that a user carries out physical activities during a specific period of time. The physical performance can be represented by, for example, the calorie expenditure in a specific time period, the MET (metabolic equivalent of task), or the like.

In the present disclosure, the term “physical activity data” refers to the data collected by at least one sensor/sensing unit when the user carries our physical activities. The sensor/sensing unit may include, but not limited to, a heart rate sensor to detect the user's heart rate, an accelerometer sensor to detect the motion of the user, and the like.

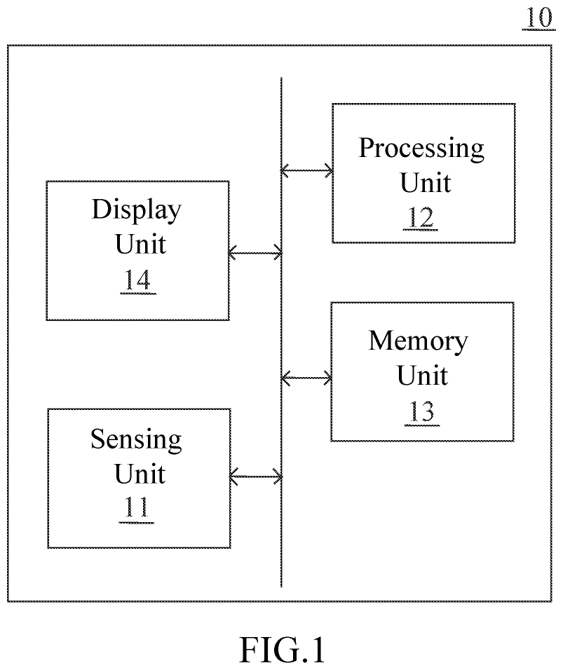

The method in the present invention may be applied in the devices such as a measurement system, a device worn on the user (e.g., the device attached to the wrist belt or chest belt), a wrist top device, a mobile smart device, a portable device, a personal computer, a server, or a combination thereof. FIG. 1 illustrates a schematic block diagram of an exemplary device 10 in the present invention. The device 10 may comprise a sensing unit 11 (e.g., at least one sensor), a processing unit 12, a memory unit 13, and a displaying unit 14. One unit may communicate with another unit in a wired or wireless way. In one embodiment, the device 10 may consist of at least two devices including, for example, the sensing unit 11 (e.g., the device worn on the user or watch) and the processing unit 12 (e.g., mobile smart device or mobile phone). In another embodiment, the sensing unit 11 and the processing unit 12 may be in a single device 10 (e.g., the device worn on the user or the watch).

The sensing unit 11 may be attached to or comprised in a belt worn on the user. The sensing unit 11 may be a sensor (e.g., heart physical activity sensor) which may measure a signal associated with the physiological data, the cardiovascular data, or the internal workload from the user's body. The signal may be measured by applying a sensor on chest, wrist, or any other human part. The processing unit 12 may be a computing device or any suitable processing device for executing software instructions, such as a central processing unit (CPU). In one embodiment, the device may consist of at least two devices, including, for example, a first portion of the computing unit in one device (e.g., the device worn on the user or watch) and a second portion of the computing unit in another device (e.g., mobile smart device or mobile phone), and the first portion of the computing unit may communicate with the second portion of the computing unit in a wired or wireless way. In another embodiment, the first portion of the computing unit and a second portion of the computing unit may be in a single device (e.g., the device worn on the user or watch).

The memory unit 13 may include random access memory (RAM) and read only memory (ROM), but it is not limited to this case. The memory unit 13 may include any suitable non-transitory computer readable medium, such as ROM, CD-ROM, DVD-ROM, and so on. Also, the non-transitory computer readable medium is a tangible medium. The non-transitory computer readable medium includes a computer program code which, when executed by the processing unit 12, causes the device 10 to perform desired operations.

The display unit 14 may be a display for providing a physical activity suggestion in the physical activity duration 21 to improve the physical performance of the user to gradually meet a plurality of target values of the physical performance set in the physical activity duration 21.

The sensing unit 11, the processing unit 12, the memory unit 13, and the displaying unit 14 in the device 10 may have any suitable configuration and it doesn't be described in detail therein.

The disclosure discloses a method for improving a physical performance of a user. The physical performance may be determined based on physical activity data by using at least one sensor. The method comprises providing, by a processing unit 12, a physical activity suggestion in a physical activity duration to improve the physical performance of the user to gradually meet a plurality of target values of the physical performance set in the physical activity duration.

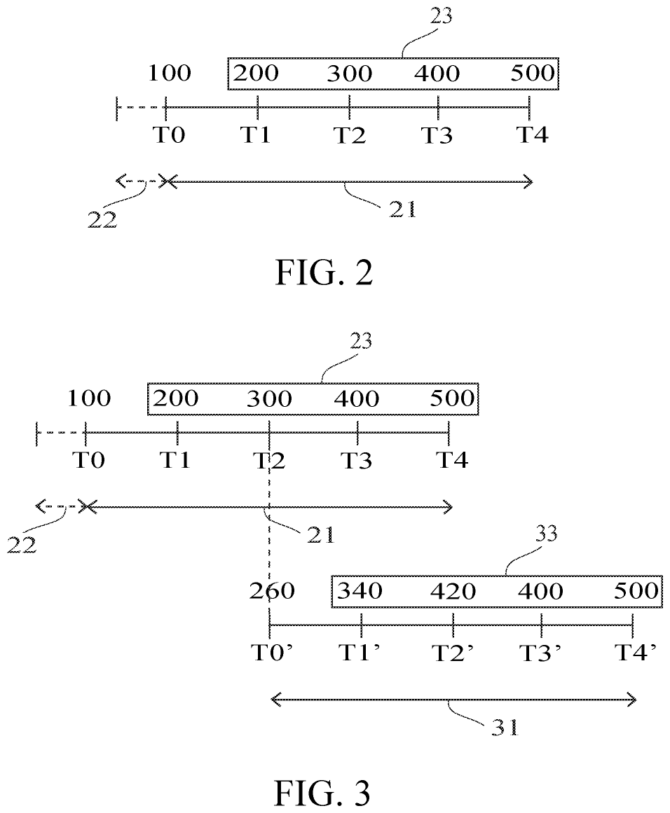

FIG. 2 illustrates an embodiment of the physical activity duration 21 when the method for improving the physical performance of the user is applied. The physical performance may be represented by physiological/action ability such as VO₂ₘₐₓ or energy expenditure such as calories or MET (Metabolic Equivalent of Task). The physical activity duration 21 is from a beginning time T0 to a final target time T4 and comprise middle targets T1, T2, and T3. In an embodiment, the reference value of the physical performance is an initial value of the physical performance determined in the beginning time T0 of the physical activity duration 21. In another embodiment, the reference value of the physical performance may be a median value of the physical performance determined in the median time of the physical activity duration 21. The reference values may be determined based on the physical performance of the user in many ways and not limited to the aforementioned embodiments.

The initial value of the physical performance of the user may be determined in the beginning time T0 of the physical activity duration 21 based on the corresponding physical activity data in a duration 22 with a specific period of time before the beginning time T0 of the physical activity duration 21. In one embodiment, the reference (initial) value of the physical performance of the user may be input by the user or the person (e.g., coach or trainer) associated with the user. In the embodiments of the physical activity duration 21 of FIG. 2, the physical performance of the user has an initial value, for example, 100 MET.

After the initial value and the final target time are determined, a plurality of target values, for example 200, 300, 400, and 500 MET in the frame 23 in FIG. 2, of the physical performance follow the reference (initial) value of the physical performance of the user in the physical activity duration 21 are determined based on the initial values and the target time. The duration between the adjacent target values may be the same or different; preferably, the duration between the adjacent target values is the same.

The target values of the physical performance may be set in increasing order in the physical activity duration 21. In one embodiment, all the target values of the physical performance are set in increasing order in the physical activity duration 21, as shown in FIG. 2.

In another embodiment, some of the target values of the physical performance may be arranged in increasing order in the physical activity duration 21 and some are not. In first example of the embodiment, the target values of the physical performance are 200, 300, 300, and 400 MET, and the purpose of the first 300 and the second 300 MET are used for maintenance. In second example of the embodiment, the target values of the physical performance are 200, 310, 290, and 400 MET, and the purpose of the target values of 310 and 290 MET are used for recovery.

In still another embodiment, the target values of the physical performance may be determined based on the reference (initial) value and the final/largest value of the physical performance. For example, the final/largest value of 500 MET of the target values of the physical performance is determined and then the remaining target values of 200, 300, and 400 MET are determined, for example, by dividing the difference between the final value of 500 MET and the initial value of 100 MET (i.e., 400 MET) into equal parts.

The physical activity suggestion in the physical activity duration 21 can be provided to improve the physical performance of the user to gradually meet the target values of the physical performance. In one embodiment, the physical activity suggestion including different physical activity plans respectively corresponding to the target values. The physical activity plan may include words, a voices, images, or videos. Finally, the final/largest value of the target values can be met. The physical activity suggestion may comprise a physical activity instruction to lead the user, such as a plan of running, walking, weight training, aerobic exercises, or the like.

The disclosure also discloses a modified method for improving the physical performance of the user if one of the target values of the physical performance is not met in the physical activity duration 21. The method further comprises a step of determining whether the target value is met according to the physical activity data collected during the physical activity duration 21 and before the target time corresponding to the target value, i.e., the physical activity data collected between the target time corresponding to the target value and the previous target time. In response to one of the target values of the physical performance is not met at the corresponding target time in the physical activity duration 21, a new modified physical activity duration with modified reference value and at least one modified target value and corresponding target time is determined based on the collected physical activity data in a performed portion of the physical activity duration 21 and provided to the user. In an embodiment, the method further comprises providing a modified physical activity suggestion in the modified physical activity duration to the user to increase the physical performance and gradually meet the modified target value of the physical performance at the corresponding modified target time.

FIG. 3 illustrates an embodiment of the modified physical activity duration 31 when the modified method for improving the physical performance of the user is applied. The modified physical activity duration 31 is from a new beginning time T0′ to a new final target time T4′ and comprise new middle targets T1′, T2′, and T3′. In an embodiment, the modified initial value of the physical performance at the beginning time T0′ may be determined based on the collected physical activity data before the target time T2 or between the target times T1 and T2 in the physical activity duration 21, the disclosure is not limited to this case.

For example, in FIG. 3, the target value of the physical performance is 300 MET at time T2 of the physical activity duration 21, but the actual value of the physical performance measured at time T2 is 260 MET; because the actual value of the physical performance is lower than the target value 300 MET of the physical performance at target time T2, a new modified physical activity duration 31 with new modified target times from time T0′ to time T4′ and new modified target values 340, 420, 400, and 500 in the frame 33 of the physical performance is determined and provided to the user with modified physical activity suggestion. In an embodiment, the modified initial value at T0′ may be determined based on the corresponding physical activity data in the performed portion of the physical activity duration 21, for example, the actual value 260 MET of the physical performance in the performed portion of the physical activity duration 21.

In an embodiment, the final/largest target value 500 MET at time T4′ of the physical performance in the modified physical activity duration 31 is still the same as the final/largest target value 500 MET at time T4 of the physical performance in the physical activity duration 21, but the difference between the adjacent modified target values in the modified physical activity duration 31 (from time T0′ to time T4′) is smaller than the difference between the adjacent target values in the physical activity duration 21 (from time T0′ to time T4′), the disclosure is not limited to this case.

In another embodiment, the final/largest target value of the physical performance in the modified physical activity duration 31 (from time T0′ to time T4′) may be set to be lower than the final/largest target value of the physical performance in the physical activity duration 21 (from time T0 to time T4) if the original final target value 500 MET is too difficult to be achieved by the user.

In an embodiment, the duration between the adjacent modified target values, for example 340, 420, 400, 500 in the frame 33 in FIG. 3, may be the same or different.

In an embodiment, if the physical performance determined at the target time is higher than the corresponding target value of the physical performance, the target value of the physical performance is met. In another embodiment, if the physical performance is within a tolerance associated with the target value of the physical performance, the target value of the physical performance is also met.

The above disclosure is related to the detailed technical contents and inventive features thereof. People skilled in the art may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for improving physical performance of a user, adapted to a device, the method comprising:
determining a first reference value of the physical performance of the user in a first reference time point of a first physical activity duration and a final target value of the physical performance corresponding to a final target time, wherein the first reference value is determined based on a collected first physical activity data;
determining a plurality of middle target values of the physical performance arranged in increasing order in the first physical activity duration based on the first reference value, the final target value, and the final target time, wherein the plurality of middle target values of the physical performance respectively corresponds to a plurality middle target times; and
providing a first physical activity suggestion to meet the target values of the physical performance.

2. The method of claim 1, wherein the first reference value is an initial value, the first reference time point of the first physical activity duration is a starting point of the first physical activity duration, wherein the initial value is determined based on the collected first physical activity data collected before the starting point.

3. The method of claim 1, wherein the first reference value, the final target value, and the plurality of the middle target values of the physical performance is represented by MET (Metabolic Equivalent of Task).

4. The method of claim 1, wherein the first physical activity suggestion includes different physical activity plans respectively corresponding to the target values.

5. The method of claim 1, further comprising:
determining whether a first target value is met according to a second physical activity data collected between the first target time corresponding to the first target value and a previous target time, wherein the first target value is one of the middle target values and the final target value.

6. The method of claim 5, further comprising:
in response to the determination that the first target value is not met at the first target time, determining a modified second physical activity duration with a second reference value and at least one modified target value and corresponding target time based on a third physical activity data collected in a performed portion of the first physical activity duration.

7. The method of claim 6, further comprising:
determined a modified second physical activity suggestion to meet the modified target values, and providing to the user.

8. The method of claim 5, further comprising:
receiving a modified final target value from an input of the user.

9. A device for improving physical performance of a user, comprising:
a memory unit, storing a program code; and
a processing unit, coupled to the memory unit, and accessing the program code to execute:
determining a first reference value of the physical performance of the user in a first reference time point of a first physical activity duration and a final target value of the physical performance corresponding to a final target time, wherein the first reference value is determined based on a first physical activity data collected by a sensing unit;
determining a plurality of middle target values of the physical performance arranged in increasing order in the first physical activity duration based on the first reference value, the final target value, and the final target time, wherein the plurality of middle target values of the physical performance respectively corresponds to a plurality middle target times; and
providing a first physical activity suggestion by a displaying unit to meet the target values of the physical performance.

10. A non-transitory computer readable storage medium, the non-transitory computer readable storage medium recording an executable computer program, and the executable computer program being loaded to a device for improving the physical performance of a user to execute:
determining a first reference value of the physical performance of the user in a first reference time point of a first physical activity duration and a final target value of the physical performance corresponding to a final target time, wherein the first reference value is determined based on a collected first physical activity data;
determining a plurality of middle target values of the physical performance arranged in increasing order in the first physical activity duration based on the first reference value, the final target value, and the final target time, wherein the plurality of middle target values of the physical performance respectively corresponds to a plurality middle target times; and
providing a first physical activity suggestion to meet the target values of the physical performance.
